(19) United States
(12) Patent Application Publication
(10) Pub. No.: US 2026/0234332 A1
(43) Pub. Date: Aug. 13, 2026

(54) POLYCYCLIC DIANHYDRIDE AND ACID ANHYDRIDE COMPOUNDS HAVING SULFONYL GROUP, AND POLYIMIDE-BASED RESIN AND FILM USING THE SAME

(71) Applicant: KOREA ADVANCED INSTITUTE OF SCIENCE AND TECHNOLOGY, Daejeon (KR)

(72) Inventors: SangYoul Kim, Daejeon (KR); Tae Joon Byeon, Daejeon (KR); Da-sol Park, Daejeon (KR)

(21) Appl. No.: 19/425,637

(22) Filed: Dec. 18, 2025

(30) Foreign Application Priority Data
Feb. 10, 2025 (KR) 10-2025-0016497
Apr. 18, 2025 (KR) 10-2025-0050851

(51) Int. Cl. C08G 73/10; C07D 497/04

(52) U.S. Cl. CPC C08G 73/1085; C07D 497/04

(57) ABSTRACT

Provided are polycyclic dianhydride and acid anhydride compounds having a sulfonyl group, a method for preparing dianhydride and acid anhydride compounds, and polyimide-based resin and film having excellent heat resistance, flexibility, and transparency, including a repeating unit of the dianhydride and acid anhydride compounds.

### CROSS-REFERENCE TO RELATED APPLICATIONS

This application claims priority under 35 U.S.C. § 119 to Korean Patent Application No. 10-2025-0016497, filed on Feb. 10, 2025, and Korean Patent Application No. No. 10-2025-0050851, filed on Apr. 18, 2025, in the Korean Intellectual Property Office, the disclosures of which are incorporated herein by reference in their entireties.

### TECHNICAL FIELD

The following disclosure relates to polycyclic dianhydride and acid anhydride compounds, and polyimide-based resin and film having excellent heat resistance, flexibility, and transparency, prepared therefrom.

### BACKGROUND

Recently, as display devices have been developed in various forms, thinning and variability in the form of being folded or rolled have become important, and attempts to replace a conventional glass substrate with a polyimide-based film which is light and flexible and has excellent chemical resistance and the like are continuing. Since flexible displays are light, are unbreakable, and have no restriction on shape, they allow various designs and have no limitation in terms of portability or size, and thus, display devices having various forms may be manufactured. A polymer material for a substrate which may replace a glass material substrate should have physical, thermal, and optical properties at similar levels to glass. Though polyimide resin has excellent physical properties such as heat resistance, it is essential to improve inherent yellow index characteristics without deterioration of heat resistance and dimensional stability and impart colorless and transparent optical properties, in order to apply a polyimide-based film to a display device.

In particular, since low-temperature polycrystalline oxide (LTPO) and organic light-emitting diode (OLED) display devices which are in increasing demand recently involve a high temperature process such as deposition and annealing of a thin film transistor (TFT) and organic materials, they should not be deformed even in a high-temperature process. As an example, they require thermal properties such as a low coefficient of thermal expansion at a similar level to the organic material and metal used in TFT and a high glass transition temperature of 400° C. or higher.

However, when it is intended to impart transparency to the polyimide-based film, it generally results in weakened heat resistance and an increased coefficient of thermal expansion. Though studies for developing a polyimide-based film having high heat resistance and low coefficient of thermal expansion, and being colorless and transparent have been continued, this is considered as being a very difficult technical problem.

### RELATED ART DOCUMENTS

### Patent Document

Korean Patent Laid-Open Publication No. 10-2021-0003100 (Jan. 11, 2021)

### SUMMARY

An embodiment of the present invention is directed to providing polycyclic dianhydride and acid anhydride compounds having a sulfonyl group, and a method for preparing the same.

Another embodiment of the present invention is directed to providing novel polyimide-based resin and film having excellent heat resistance, flexibility, and transparency, including repeating units of the dianhydride and acid anhydride compounds.

Still another embodiment of the present invention is directed to providing a flexible display device including the polyimide-based film.

In one general aspect, a dianhydride compound of the following Chemical Formula 1 and an acid anhydride compound of the following Chemical Formula 2 are provided:

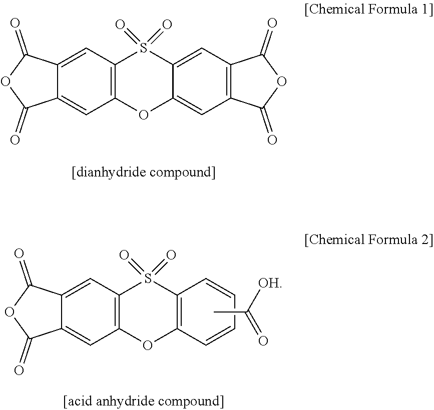

In another general aspect, a method for preparing the compound of Chemical Formula 1 includes oxidizing a precursor of the following Chemical Formula 25, in the presence of an oxidizing agent. In addition, a method for preparing the compound of Chemical Formula 2 includes oxidizing a precursor of the following Chemical Formula 26, in the presence of an oxidizing agent:

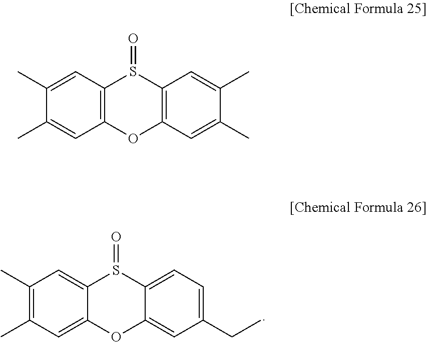

In another general aspect, a polyimide-based resin includes a repeating unit represented by the following Chemical Formula 3 or 4:

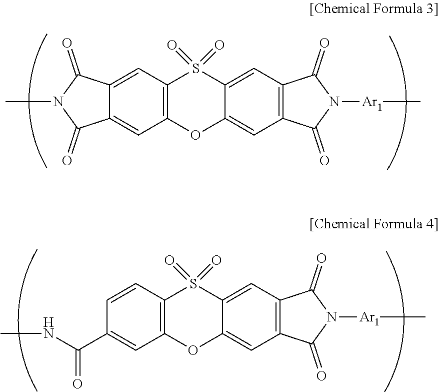

wherein Ar₁ is independently of each other

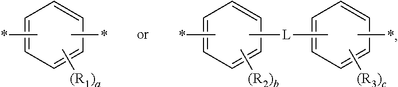

and
L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2.

The polyimide-based resin of the present invention may include a repeating unit selected from the following Chemical Formulae 5 to 8:

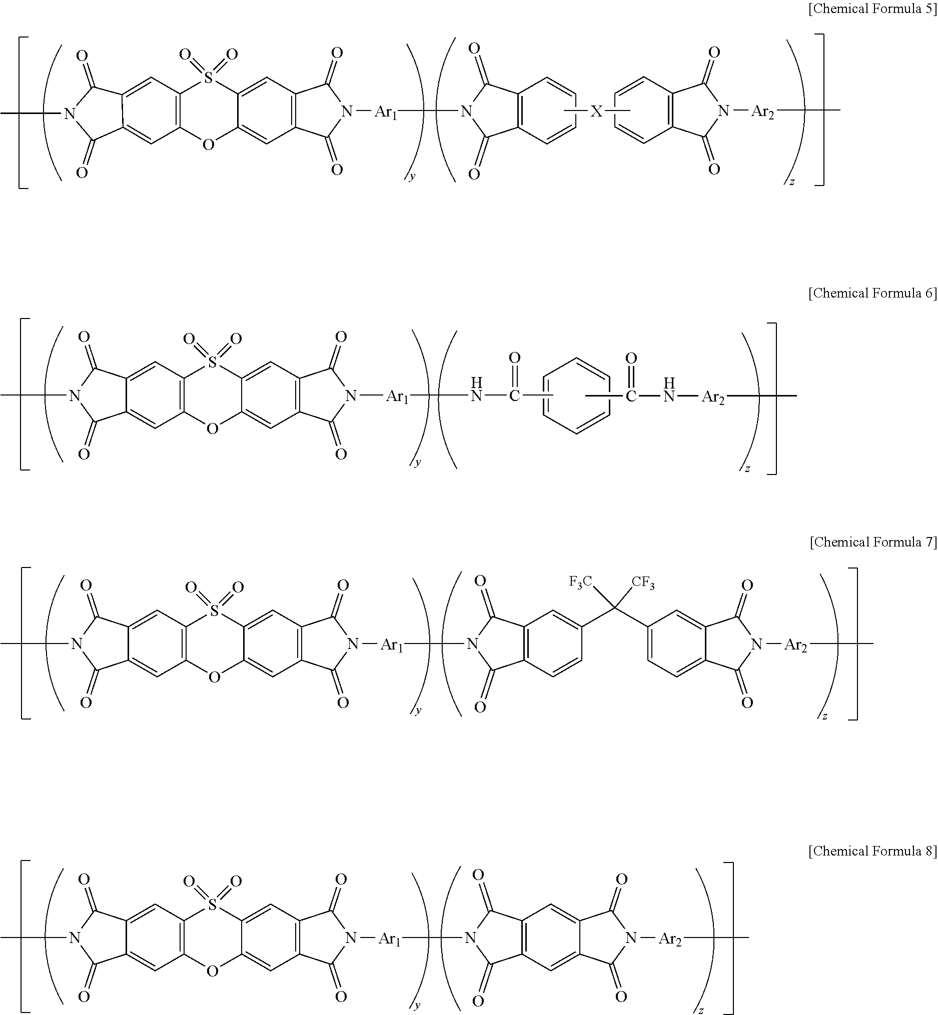

wherein X is a single bond, —CH₂—, —O—, —S—, —SO₂—, —CO—, isopropylidene, hexafluoroisopropylidene, or a combination thereof,
Ar₁ and Ar₂ are independently of each other

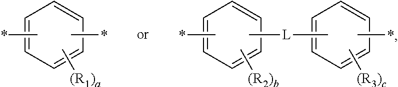

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In an exemplary embodiment, the polyimide-based resin of the present invention may include a repeating unit represented by the following Chemical Formulae 9 to 11:

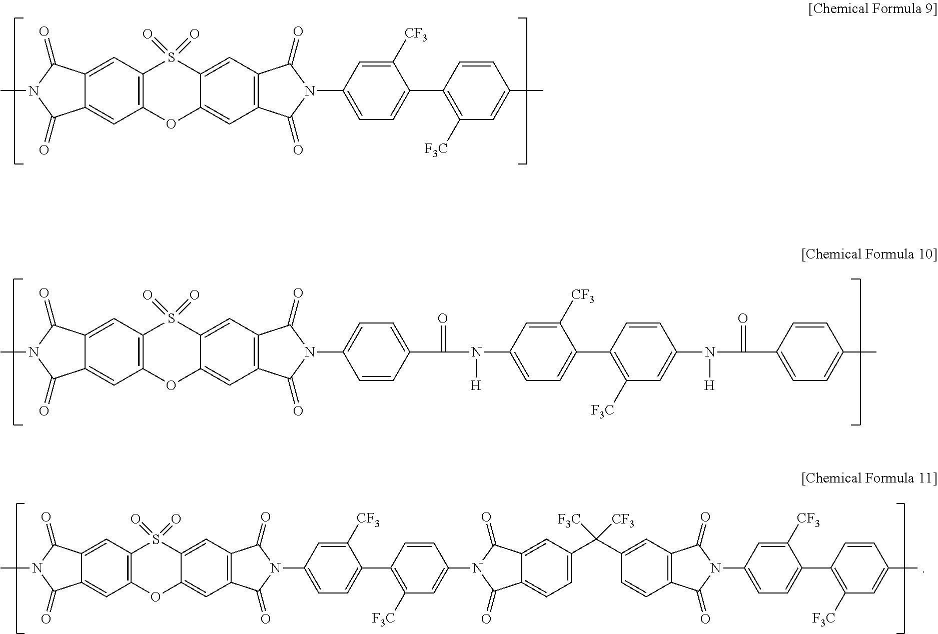

The polyimide-based resin according to an exemplary embodiment of the present invention may include a repeating unit represented by the following Chemical Formula 12:

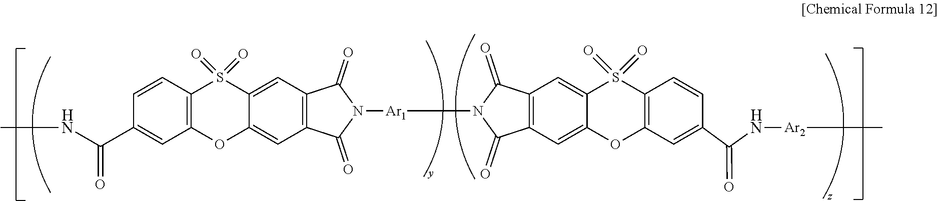

wherein Ar₁ and Ar₂ are independently of each other

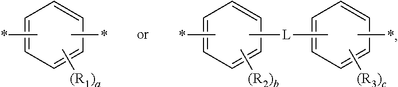

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; a, b, and c are independently of one another an integer of 0 to 2, and
y:z is 100:0 to 10:90.

In an exemplary embodiment, the polyimide-based resin may have a number average molecular weight of 10,000 to 200,000 g/mol.

The polyimide-based resin according to an exemplary embodiment of the present invention may be prepared by: reacting a dianhydride compound represented by the following Chemical Formula 1 and an aromatic diamine represented by the following Chemical Formula 21; sequentially reacting a dianhydride compound represented by the following Chemical Formula 23 and an aromatic diamine represented by the following Chemical Formula 22; and performing imidization so that a repeating unit represented by the following Chemical Formula 5 is included:

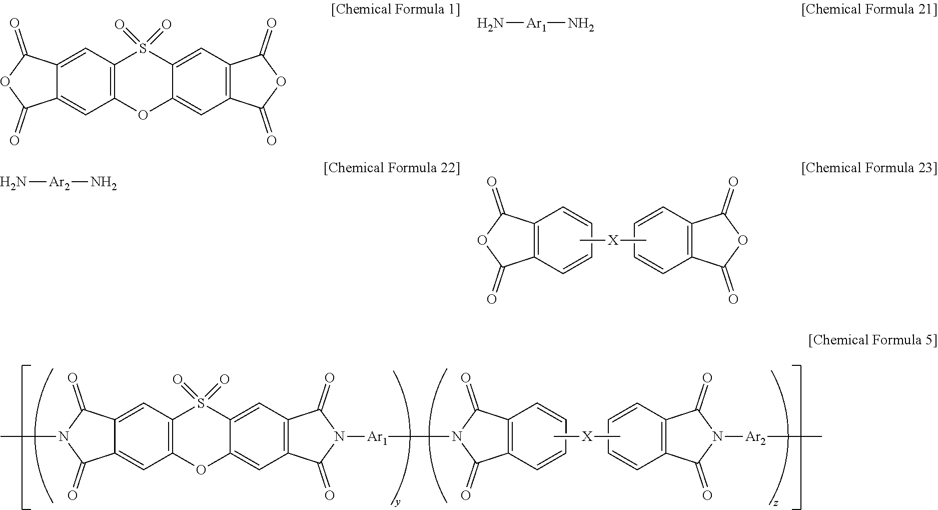

wherein X is a single bond, —CH₂—, —O—, —S—, —SO₂—, —CO—, isopropylidene, hexafluoroisopropylidene, or a combination thereof,
Ar₁ and Ar₂ are independently of each other

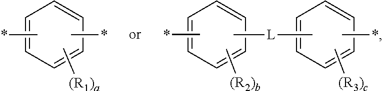

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

The polyimide-based resin according to an exemplary embodiment of the present invention may be prepared by: reacting an acid anhydride compound represented by the following Chemical Formula 2 and an aromatic diamine represented by the following Chemical Formula 21; sequentially reacting an acid anhydride compound represented by the following Chemical Formula 2 and an aromatic diamine represented by the following Chemical Formula 22; and performing imidization so that a repeating unit represented by the following Chemical Formula 12 is included:

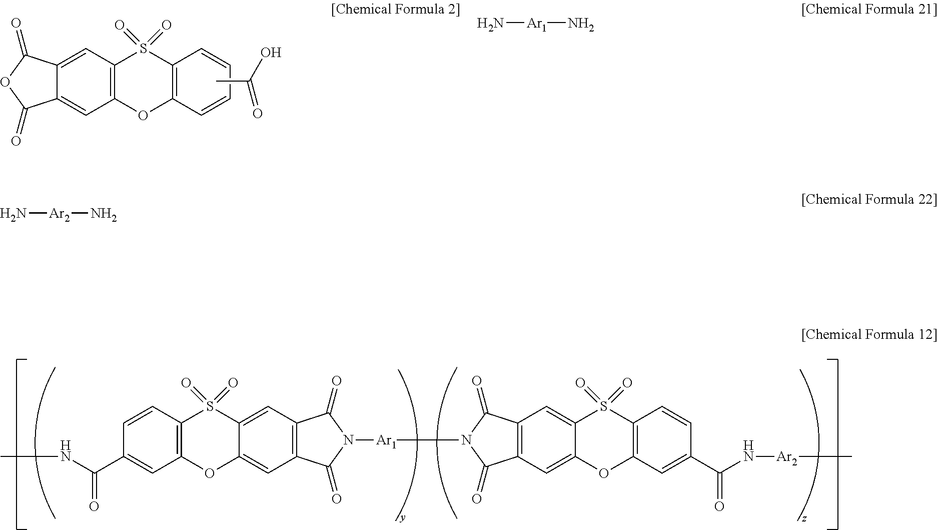

wherein Ar₁ and Ar₂ are independently of each other

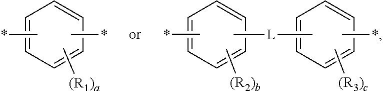

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

The polyimide-based resin according to an exemplary embodiment of the present invention may be prepared by: reacting a dianhydride compound represented by the following Chemical Formula 1 and an aromatic diamine represented by the following Chemical Formula 21; sequentially reacting an acyl chloride represented by the following Chemical Formula 24 and an aromatic diamine represented by the following Chemical Formula 22; and performing imidization so that a repeating unit represented by the following Chemical Formula 6 is included:

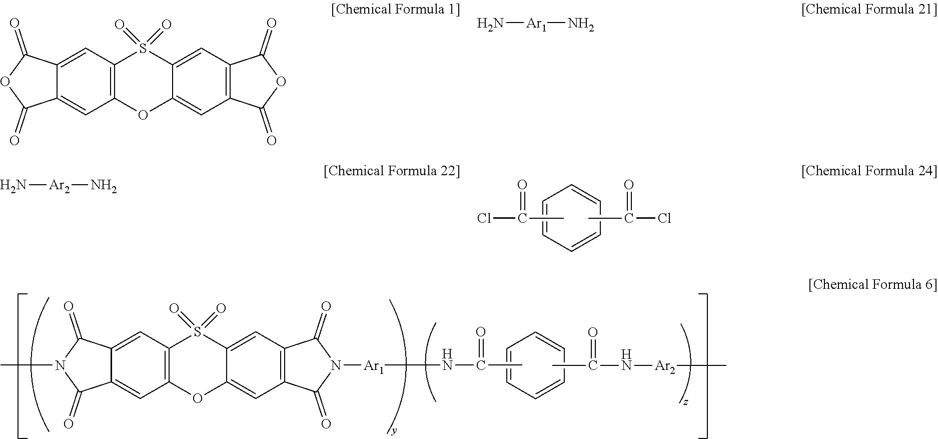

wherein Ar₁ and Ar₂ are independently of each other

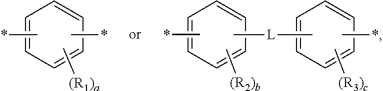

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In another general aspect, a composition for forming a polyimide-based film includes the polyimide-based resin described above.

In an exemplary embodiment, the composition for forming a polyimide-based film may further include an organic solvent.

In an exemplary embodiment, the composition for forming a polyimide-based film may include 5 to 50 wt % of the polyimide-based resin with respect to the total weight of the composition.

In another general aspect, a polyimide-based film formed from the composition for forming a film is provided.

In an exemplary embodiment, the polyimide-based film may have a glass transition temperature (T_{g}) of 400° C. or higher.

In an exemplary embodiment, the polyimide-based film may have a coefficient of thermal expansion of 20 ppm/° C. or less.

In an exemplary embodiment, the polyimide-based film may have a transmittance for a wavelength of 550 nm of 85% or more.

In still another general aspect, a flexible display device includes the polyimide-based film.

Other features and aspects will be apparent from the following detailed description, the drawings, and the claims.

### BRIEF DESCRIPTION OF THE DRAWINGS

FIG. 1 shows results of proton nuclear magnetic resonance (¹H NMR) analysis of an intermediate D-2.

FIG. 2 shows results of carbon nuclear magnetic resonance (¹³C NMR) analysis of an intermediate D-2.

FIG. 3 shows results of proton nuclear magnetic resonance (¹H NMR) analysis of an intermediate D-3.

FIG. 4 shows results of carbon nuclear magnetic resonance (¹³C NMR) analysis of intermediate D-3.

FIG. 5 shows results of proton nuclear magnetic resonance (¹H NMR) analysis of a dianhydride compound.

FIG. 6 shows results of carbon nuclear magnetic resonance (¹³C NMR) analysis of a dianhydride compound.

FIG. 7 shows results of polyamic acid proton nuclear magnetic resonance (1H NMR) analysis of Example 1.

FIG. 8 shows results of Fourier transform infrared spectroscopic (FT-IR) analysis of polyimide (PI-1) of Example 1.

FIG. 9 is a thermogravimetric analysis graph under a nitrogen environment and an air environment of polyimide (PI-1),

FIG. 10 is a differential scanning calorimetric analysis graph of polyimide (PI-1) of Example 1.

FIG. 11 is photographs of the polyimide films of Examples 1 and 3.

### DETAILED DESCRIPTION OF EMBODIMENTS

In order to sufficiently understand the configuration and effect of the present invention, preferred exemplary embodiments of the present invention will be described with reference to the attached drawings. However, the present invention is not limited to the exemplary embodiments disclosed later, but may be implemented in various forms and variously modified. Rather, the exemplary embodiments may be described so that the disclosure of the present invention is thorough and complete, and may be provided so that the scope of the present invention is fully conveyed to a person with ordinary skill in the art to which the present invention pertains. A person with ordinary skill in the art will understand that the concept of the present invention may be carried out in any appropriate environment.

The term used in the present specification is for describing the examples rather than limiting the present invention. In the present specification, unless otherwise stated in the text, a singular form also includes a plural form. The word “comprise” and/or “comprising” used herein will be understood to imply the inclusion of stated constituents, steps, operations and/or elements but not the exclusion of any other constituents, steps, operations and/or elements.

In the present specification, an alkyl group may be a linear alkyl group, a branched alkyl group, or a cyclic alkyl group. The number of carbons of the alkyl group is not particularly limited, but may be an alkyl group having 1 to 5 carbons. An example of the alkyl group may be a methyl group, an ethyl group, a n-propyl group, an isopropyl group, a n-butyl group, a t-butyl group, an i-butyl group, a 2-ethylbutyl group, a 3,3-dimethylbutyl group, a n-pentyl group, an i-pentyl group, a neopentyl group, a t-pentyl group, a cyclopentyl group, and the like, but is not limited thereto.

In the present specification, the number of carbons of an alkoxy group is not particularly limited, but may be 1 or more and 5 or less. The alkoxy group may include an alkyl alkoxy group and an aryl alkoxy group.

In the present specification, an example of a halogen may be fluorine (F), chlorine (C1), bromine (Br), iodine (I), and the like, but is not limited thereto. In the present specification, “unsubstituted or substituted” may refer to being unsubstituted or substituted by one or more substituents selected from the group consisting of a hydrogen atom, a halogen atom, a hydroxyl group, an alkoxy group, a halogenated alkyl group, a halogenated alkoxy group, an alkyl group, an alkenyl group, an aryl group, and a heterocyclic group. In addition, each of the substituents exemplified above may be unsubstituted or substituted. For example, the halogenated alkoxy group may be interpreted as an alkoxy group.

Unless otherwise defined in the chemical formula of the present specification, when a chemical bond is not drawn at a position where a chemical bond should be drawn, it means that a hydrogen atom is bonded to the position.

In the present specification, the same reference numeral throughout the specification may refer to the same constituent element.

In the present specification, “polyimide-based” may refer to all polymers in which an imide bond (—CO—N—CO) is included in the repeating unit.

Hereinafter, the present invention will be described in detail.

The present invention provides polycyclic dianhydride and acid anhydride compounds having a sulfonyl group which may produce a polyimide-based film having excellent heat resistance, flexibility, and transparency.

Specifically, the dianhydride and acid anhydride compounds may be represented by the following Chemical Formulae 1 and 2:

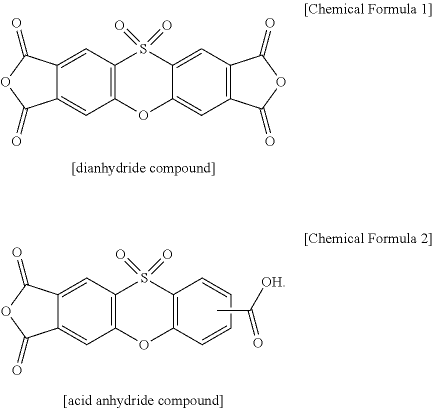

The method for preparing Chemical Formula 1 may include oxidizing a precursor of the following Chemical Formula 25, in the presence of an oxidizing agent. After the oxidization reaction of the precursor, the dianhydride may be finally obtained from the precursor, by including reacting anhydrous acetic acid. The preparation method will be described in more detail in Preparation Example 1 described later:

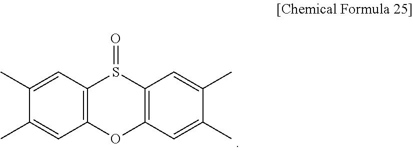

The method for preparing Chemical Formula 2 may include oxidizing a precursor of the following Chemical Formula 26, in the presence of an oxidizing agent. After the oxidation reaction of the precursor, vacuum sublimation may be performed to finally obtain the acid anhydride. The preparation method will be described in more detail in Preparation Example 2 described later:

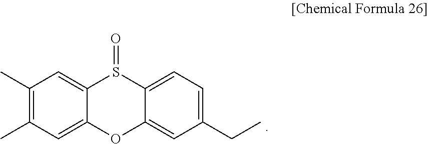 [SMILES: CCc1ccc2c(c1)Oc1cc(C)c(C)cc1S2=O]

The oxidizing agent is not particularly limited, but may use any one selected from potassium permanganate (KMnO₄), hydrogen peroxide (H₂O₂), chromic acid (H₂CrO₄), potassium sulfate (K₂S₂O₈), potassium dichromate (K₂Cr₂O₇), and the like.

The polyimide-based resin of the present invention may include a repeating unit represented by the following Chemical Formula 3 or 4:

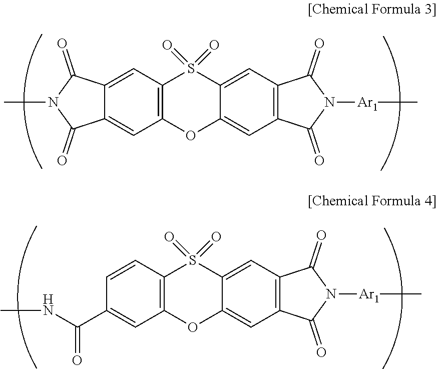

wherein Ar₁ is independently of each other

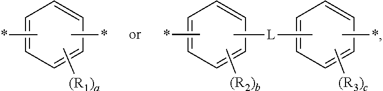

and
L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; and R₁, R₂, and R₃ are independently of one another (C1-C7)alkyl, (C1-C7)alkoxy, or halo(C1-C7)alkyl; and a, b, and c are independently of one another an integer of 0 to 2.

The polyimide-based resin of the present invention may include a repeating unit selected from the following Chemical Formulae 5 to 8:

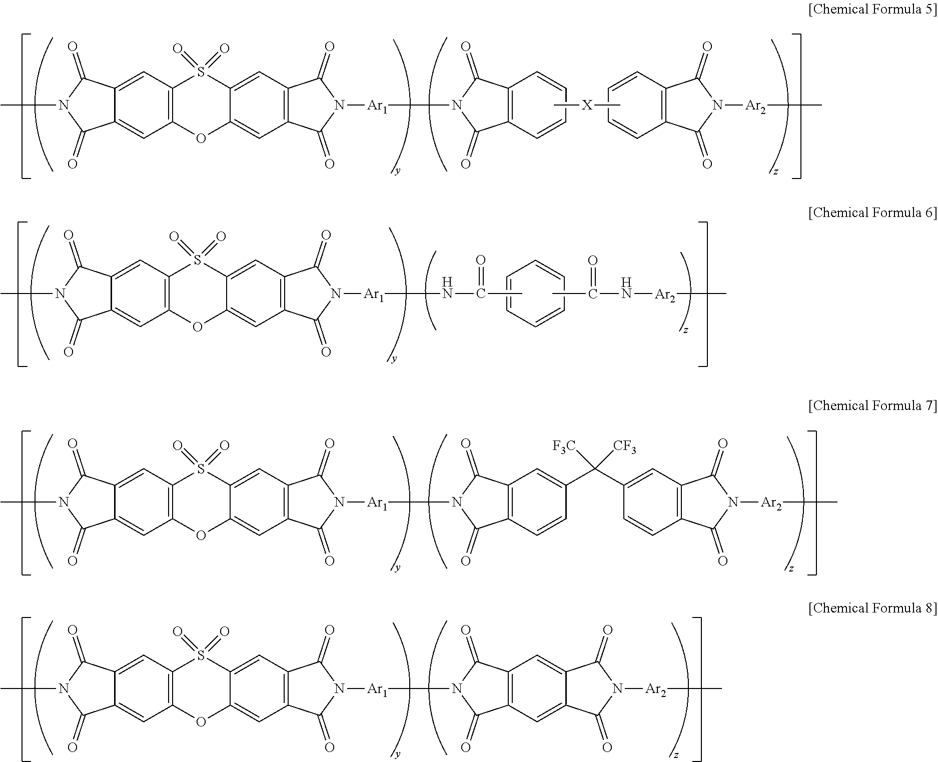

wherein X is a single bond, —CH₂—, —O—, —S—, —SO₂—, —CO—, isopropylidene, hexafluoroisopropylidene, or a combination thereof,
Ar₁ and Ar₂ are independently of each other

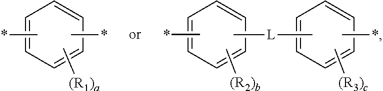

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In addition, y:z may be 90:10, 80:20, 70:30, 60:40, 50:50, 40:60, 30:70, or 20:80.

In an exemplary embodiment, Ar₁ and Ar₂ may be independently of each other selected from the following structures:

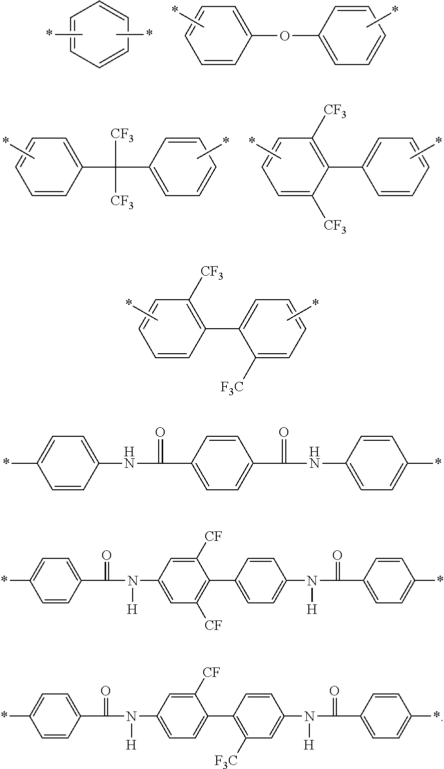

In an exemplary embodiment, the polyimide-based resin of the present invention may include a repeating unit represented by the following Chemical Formulae 9 to 11, but is not limited thereto:

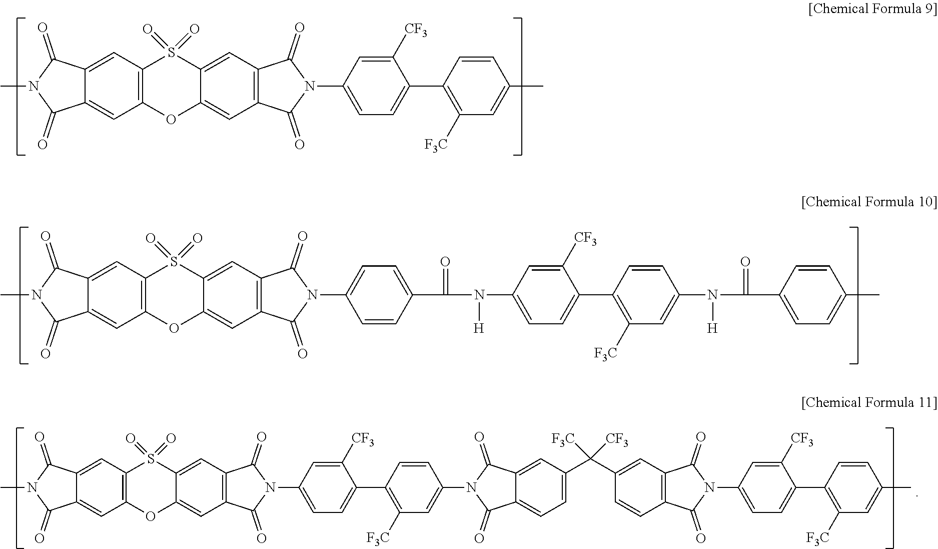

The polyimide-based resin according to an exemplary embodiment may include a repeating unit represented by the following Chemical Formula 12:

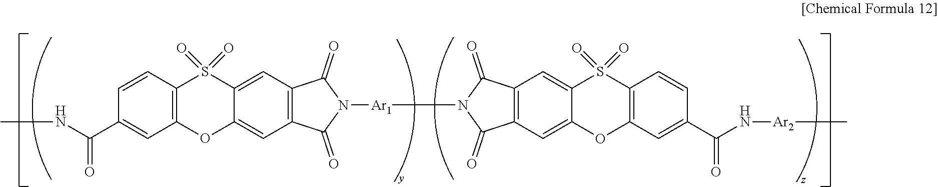

wherein Ar₁ and Ar₂ are independently of each other

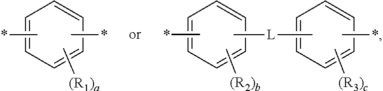

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In addition, y:z may be 90:10, 80:20, 70:30, 60:40, 50:50, 40:60, 30:70, or 20:80.

In an exemplary embodiment, the polyimide-based resin may have a number average molecular weight of 10,000 to 200,000 g/mol. The lower limit may be 10,000 g/mol or more, 20,000 g/mol or more, 30,000 g/mol or more, 50,000 g/mol or more, 100,000 g/mol or more, or 150,000 g/mol or more. The upper limit may be 200,000 g/mol or less, 180,000 g/mol or less, 160,000 g/mol or less, 130,000 g/mol or less, 110,000 g/mol or less, or 90,000 g/mol or less, and a value between the upper limit and the lower limit. For example, the number average molecular weight may be 10,000 to 200,000 g/mol, 50,000 to 200,000 g/mol, or 80,000 to 200,000 g/mol.

The polyimide-based resin according to an exemplary embodiment of the present invention may be prepared by: reacting a dianhydride compound represented by the following Chemical Formula 1 and an aromatic diamine represented by the following Chemical Formula 21; sequentially reacting a dianhydride compound represented by the following Chemical Formula 23 and an aromatic diamine represented by the following Chemical Formula 22; and performing imidization so that a repeating unit represented by the following Chemical Formula 5 is included:

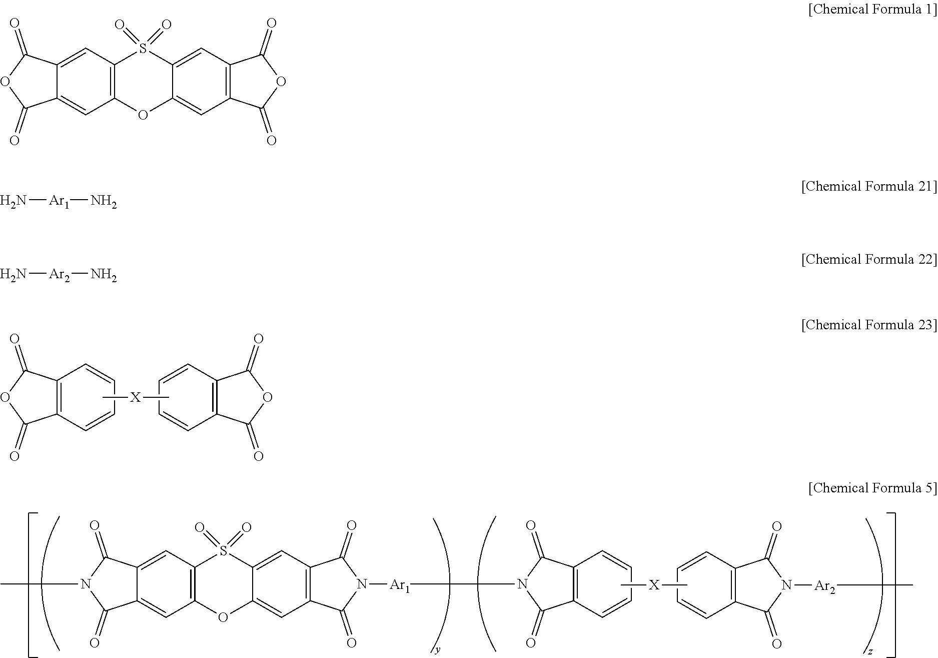

wherein X is a single bond, —CH₂—, —O—, —S—, —SO₂—, —CO—, isopropylidene, hexafluoroisopropylidene, or a combination thereof,
Ar₁ and Ar₂ are independently of each other

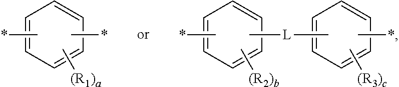

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In addition, y:z may be 90:10, 80:20, 70:30, 60:40, 50:50, 40:60, 30:70, or 20:80.

The polyimide-based resin according to an exemplary embodiment of the present invention may be prepared by: reacting an acid anhydride compound represented by the following Chemical Formula 2 and an aromatic diamine represented by the following Chemical Formula 21; sequentially reacting an acid anhydride compound represented by the following Chemical Formula 2 and an aromatic diamine represented by the following Chemical Formula 22; and performing imidization so that a repeating unit represented by the following Chemical Formula 12 is included:

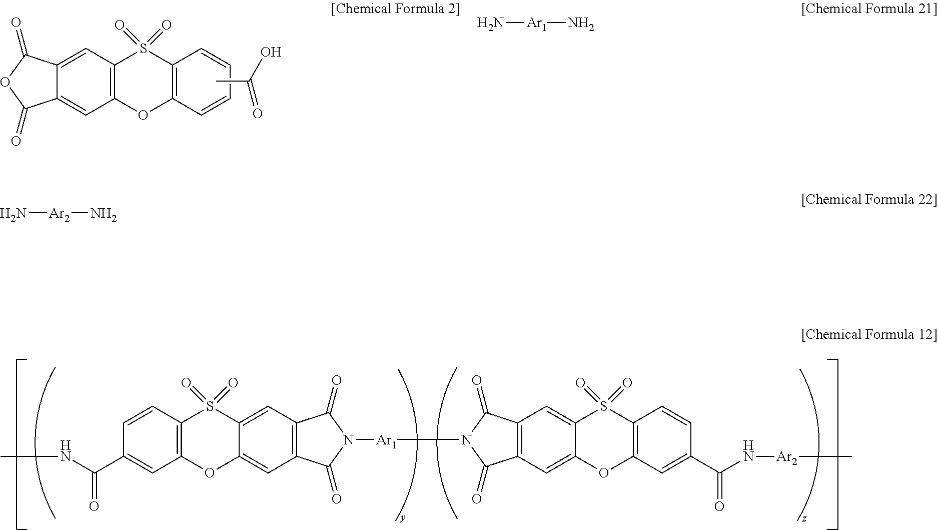

wherein Ar₁ and Ar₂ are independently of each other

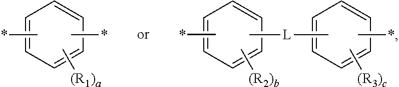

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In addition, y:z may be 90:10, 80:20, 70:30, 60:40, 50:50, 40:60, 30:70, or 20:80.

The polyimide-based resin according to an exemplary embodiment of the present invention may be prepared by: reacting a dianhydride compound represented by the following Chemical Formula 1 and an aromatic diamine represented by the following Chemical Formula 21; sequentially reacting an acyl chloride represented by the following Chemical Formula 24 and an aromatic diamine represented by the following Chemical Formula 22; and performing imidization so that a repeating unit represented by the following Chemical Formula 6 is included:

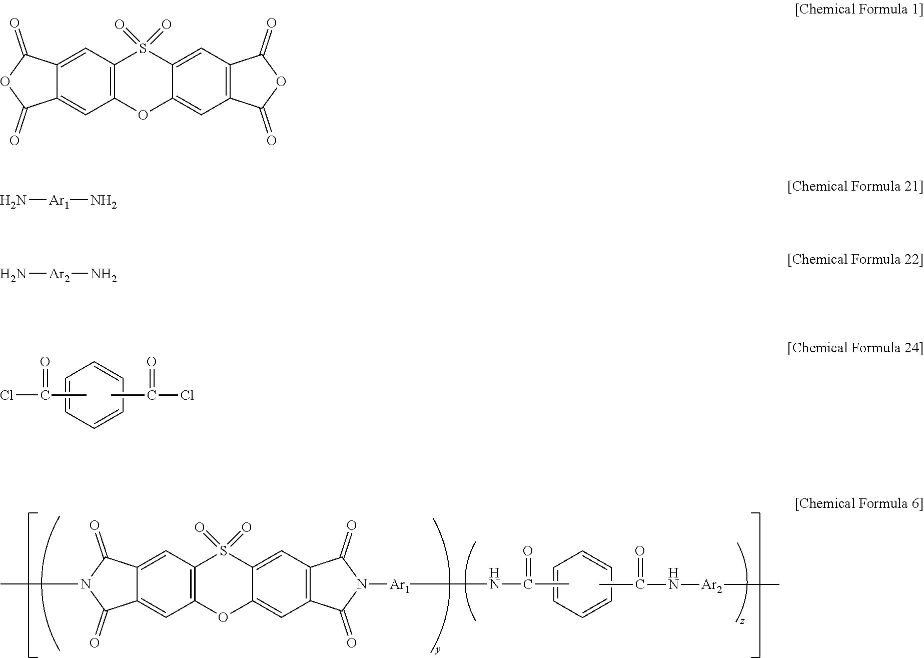

wherein Ar₁ and Ar₂ are independently of each other

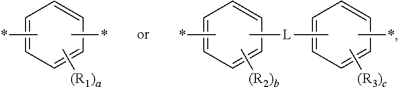

L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

In addition, y:z may be 90:10, 80:20, 70:30, 60:40, 50:50, 40:60, 30:70, or 20:80.

In another general aspect, a composition for forming a polyimide-based film includes the polyimide-based resin described above.

In an exemplary embodiment, the composition for forming a polyimide-based film may further include an organic solvent. The organic solvent may be one or two or more selected from ketones such as γ-butyrolactone, 1,3-dimethyl-2-imidazolidinone, methyl ethyl ketone, cyclohexanone, cyclopentanone, and 4-hydroxy-4-methyl-2-pentanone; aromatic hydrocarbons such as toluene, xylene, and tetramethylbenzene; glycolethers (cellosolve) such as ethylene glycol monoethyl ether, ethylene glycol monomethylether, ethylene glycol monobutyl ether, diethylene glycol monoethylether, diethylene glycol monomethylether, diethylene glycol monobutyl ether, propylene glycol monomethyl ether, propylene glycol monoethyl ether, dipropylene glycol diethyl ether, and triethylene glycol monoethyl ether; acetates such as ethyl acetate, butyl acetate, ethylene glycol monoethyl ether acetate, ethylene glycol monobutyl ether acetate, diethylene glycol monoethyl ether acetate, and dipropylene glycol monomethyl ether acetate; alcohols such as methanol, ethanol, propanol, ethylene glycol, propylene glycol, and carbitol; amides such as N,N-dimethylpropionamide (DMPA), N,N-diethylpropionamide (DEPA), N,N-dimethylacetamide (DMAc), N,N-diethylacetamide (DEAc), N,N-dimethylformamide ((DMF), N,N-diethylformamide (DEF), N-methylpyrrolidone (NMP), N-ethylpyrrolidone (NEP), and N,N-dimethylmethoxyacetamide; and the like, but is not limited thereto.

In an exemplary embodiment, the composition for forming a polyimide-based film may include 5 to 50 wt % of the polyimide-based resin with respect to the total weight of the composition. Specifically, 10 to 50 wt %, 10 to 40 wt %, 20 to 40 wt %, or 20 to 50 wt % of the polyimide-based resin may be included.

In another general aspect, a polyimide-based film formed from the composition for forming a film is provided.

The polyimide-based film may have a thickness of 1 to 500 μm, 1 to 100 μm, 1 to 50 μm, or 1 to 20 μm.

In an exemplary embodiment, the polyimide-based film may have a glass transition temperature (T_{g}) of 400° C. or higher. That is, since the polyimide-based film of the present invention has excellent thermal safety, it is not deformed even at a high-temperature process required in a flexible device and may minimize a difference in expansion and shrinkage between dissimilar materials. Specifically, the glass transition temperature (T_{g}) may be 400 to 480° C. In addition, the thermal decomposition temperature (T_{d}) may be 480° C. or higher, and though the upper limit is not particularly limited, may be 600° C. or lower.

In an exemplary embodiment, the polyimide-based film may have a coefficient of thermal expansion of 20 ppm/° C. or less. Specifically, the coefficient of thermal expansion may be 15 ppm/° C. or less, 11 ppm/° C. or less, and more preferably, 3 ppm/° C. or less. Otherwise, it may be 1 to 15 ppm/° C. or 1 to 11 ppm/° C.

In an exemplary embodiment, the polyimide-based film may have a transmittance for a wavelength of 550 nm of 85% or more. The transmittance may be 85% to 99%, specifically 85% or more, 88% or more, 89% or more, or 90% or more.

Since the polyimide-based film of the present invention may have high heat resistance and low coefficient of thermal expansion while being transparent, it is expected to be applicable as a flexible display device involved in a high-temperature process.

Hereinafter, the exemplary embodiments described above will be described in more detail through the following examples. However, the following examples are only for better understanding and do not limit the scope of a right.

### [Measurement Method of Physical Properties]

### (1) Number Average Molecular Weight

It was measured using gel permeation chromatography (GPC). 10 μm of PLgel MIXED-B as a column, polystyrene as a standard sample, and tetrahydrofuran as a solvent were used, a sample at a concentration of 5 mg/10 mL was prepared under the conditions of a temperature of 30° C. and a flow rate of 1.0 mL/min and supplied in an amount of 200 μL, and then measurement was performed.

### (2) Yellow Index

It was measured according to ASTM D1925.

### (3) Cut Off Wavelength

A transmittance was measured in accordance with ASTM D1003, and a wavelength at which the transmittance began to appear was set as a cut-off wavelength (λ₀) of each film.

### (4) Transmittance

The transmittance (T_{550 nm}) for a wavelength of 550 nm was measured in accordance with ASTM D1003.

### (5) Thermal Decomposition Temperature (T_{d}, 5%)

A thermogravimetric analysis (TA instruments, TGA Q50) method was used. The measurement conditions were a temperature range of room temperature to 800° C., a heating rate of 5° C./min, and a pressure of 1.5 bar/min with a nitrogen gas injection, and a temperature at which a mass decrease of 5% occurred was set as a thermal decomposition temperature (T_{d}, 5%).

### (6) Glass Transition Temperature (T_{g})

A second obtained value from heating and cooling performed at 10° C./min from 0° C. to 400° C. using an isothermal differential scanning calorimetry (TA instruments, DSC Q20) was measured.

### (7) Coefficient of Thermal Expansion (CTE)

It was measured using a thermomechanical analyzer (TA instruments, TMA-Q400). The measurement was performed with a load of 0.01 N at a heating rate of 5° C./min in a temperature range of room temperature to 450° C. and the expansion rates were averaged.

### [Preparation Example 1] Preparation of Dianhydride Compound

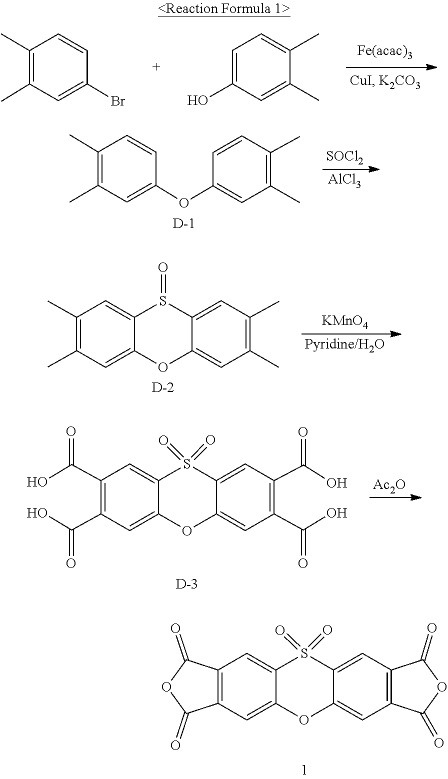

4-Bromo-1,2-xylene (6.00 g, 32.4 mmol), 3,4-dimethylphenol (4.99 g, 40.8 mmol), iron (III) acetylacetonate (Fe(acac)₃) (2.22 g, 6.29 mmol), potassium carbonate (11.30 g, 81.8 mmol), copper iodide (I) (1.20 g, 6.30 mmol), and dimethylformamide (DMF, 60.0 ml) were added to a three-neck round bottom flask to obtain a reaction solution, which was stirred at 135° C. for 12 hours. The reaction mixture was filtered through a filter paper, washed with a brine, extracted with hexane, and then dried with anhydrous magnesium sulfate. The mixture was purified with silica gel column chromatography using methane dichloride (MC)/hexane (v/v=1/5) as a development agent and dried under reduced pressure to obtain a final product D-1 which was a white solid powder (4.80 g, 66%).

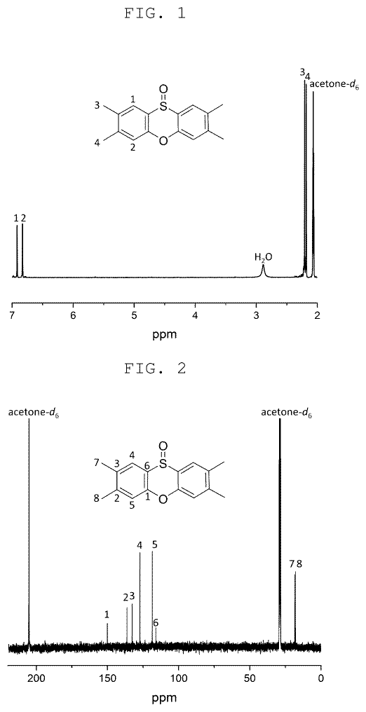

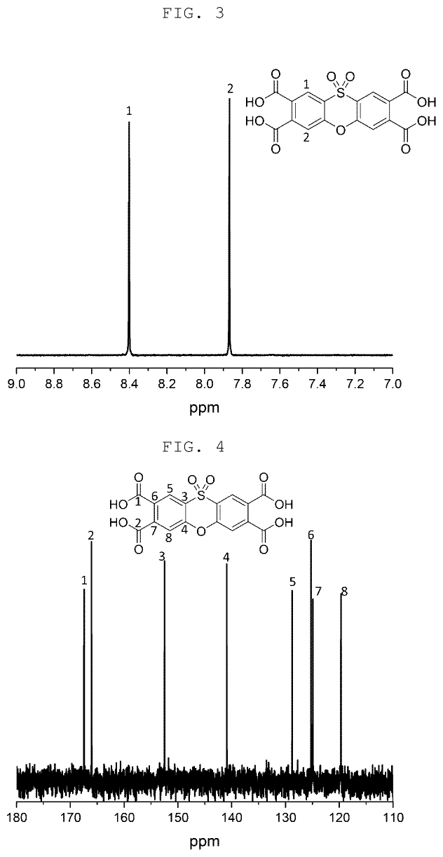

A three-neck round bottom flask containing aluminum chloride (2.82 g, 21.2 mmol), thionyl chloride (3.03 g, 25.5 mmol), and anhydrous methane dichloride (MC, 105 ml) was placed in an ice water bath and cooled. Compound D-1 (4.80 g, 21.2 mmol) was dissolved in MC, and slowly injected into the flask containing the mixture under a nitrogen flow. The reaction mixture was stirred at 0° C. for 1 hour under the nitrogen flow, and then washed with ice water, and an organic layer was dried with anhydrous magnesium sulfate. The mixture was purified with silica gel column chromatography using methane dichloride (MC)/hexane (v/v=1:20) as a development agent and dried under reduced pressure to obtain a product D-2 which was a white solid powder (1.18 g, 20.5%). ¹H NMR (400 MHz, acetone-d₆, 25° C., ppm): δ 6.90 (s, 1H), 6.81 (s, 1H), 2.19 (s, 3H), 2.16 (s, 3H). (see FIGS. 1 and 2)

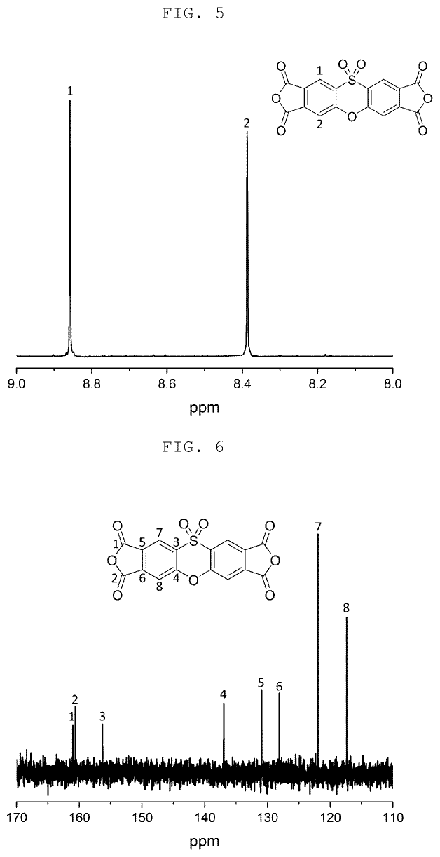

A solution of potassium permanganate (42.5 g, 268.8 mmol) dissolved in distilled water (48 ml) was divided into 6 parts and added to a three-neck round bottom flask containing Compound D-2 (6.60 g, 24.3 mmol) and pyridine (260 ml) every 30 minutes while increasing the temperature each time, and finally the temperature reached 90° C. The reaction mixture was stirred at 90° C. for 15 hours. The reaction mixture was filtered through a filter paper and washed with hot water/pyridine (v/v=3:1), and then the filtrate was concentrated under reduced pressure. The obtained product was added to the same round bottom flask, sodium hydroxide (4.88 g, 122.0 mmol), potassium permanganate (42.5 g, 268.8 mmol), and distilled water (170 ml) were added, and the reaction mixture was refluxed for 3 hours. The reaction was terminated by adding methanol to the reaction mixture, filtration was performed with a filter paper, and the filtrate was acidified by sulfuric acid to obtain a solid product, which was filtered through a filter. The final product was dried under reduced pressure to obtain a solid powder product D-3 (4.30 g, 43.1%). ¹H NMR (400 MHz, DMSO-d₆, 25° C., ppm): δ 8.40 (s, 1H), 7.87 (s, 1H). (see FIGS. 3 and 4)

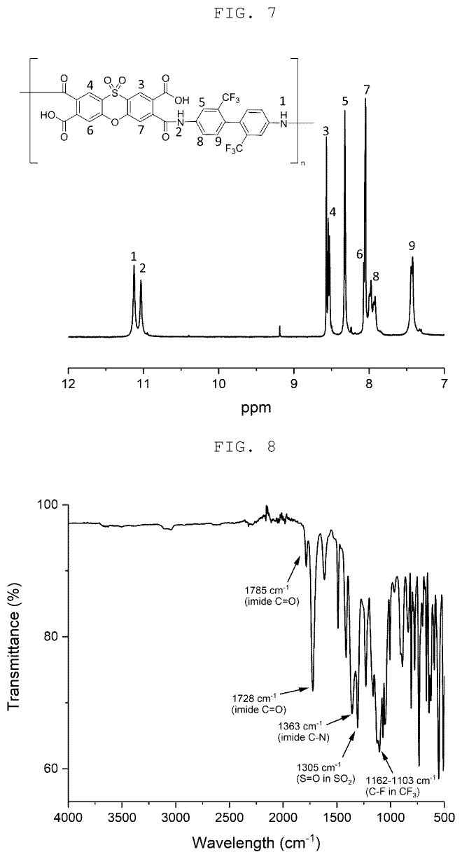

Compound D-3 (2.45 g, 6.0 mmol) and anhydrous acetic acid (25.0 ml) were added to a round bottom flask, and stirring was performed at 85° C. for 6 hours. The reaction mixture was filtered through a filter, washed with anhydrous acetic acid and toluene, and dried under reduced pressure to obtain a dianhydride compound 1 which was a white solid powder (2.0 g, 89.6%). ¹H NMR (400 MHz, acetone-d₆, 25° C., ppm): δ 8.86 (s, 1H), 8.39 (s, 1H). (see FIGS. 5 and 6)

### [Preparation Example 2] Preparation of Acid Anhydride Compound

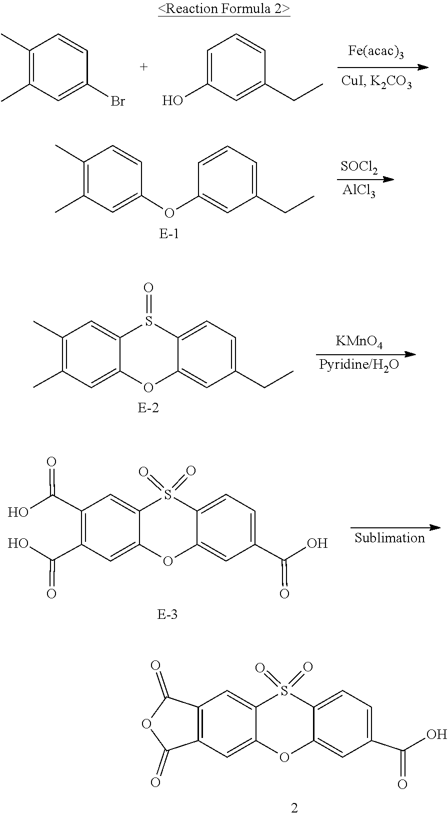

4-Bromo-1,2-xylene (6.00 g, 32.4 mmol), 3-ethylphenol (4.98 g, 40.8 mmol), iron (III) acetylacetonate (Fe(acac)₃) (2.22 g, 6.29 mmol), potassium carbonate (11.30 g, 81.8 mmol), copper iodide (I) (1.20 g, 6.30 mmol), and dimethylformamide (DMF, 60.0 ml) were added to a three-neck round bottom flask to obtain a reaction solution, which was stirred at 135° C. for 12 hours. The reaction mixture was filtered through a filter paper, washed with a brine, extracted with hexane, and then dried with anhydrous magnesium sulfate. The mixture was purified with silica gel column chromatography using methane dichloride (MC)/hexane (v/v=1/5) as a development agent and dried under reduced pressure to obtain a final product E-1 which was a white solid powder (4.60 g, 63%). A three-neck round bottom flask containing aluminum chloride (2.82 g, 21.2 mmol), thionyl chloride (3.03 g, 25.5 mmol), and anhydrous methane dichloride (MC, 105 ml) was placed in an ice water bath and cooled. Compound E-1 (4.60 g, 20.3 mmol) was dissolved in MC, and slowly injected into the flask containing the mixture under a nitrogen flow. The reaction mixture was stirred at 0° C. for 1 hour, and then washed with ice water, and an organic layer was dried with anhydrous magnesium sulfate. The mixture was purified with silica gel column chromatography using methane dichloride (MC)/hexane (v/v=1:20) as a development agent and dried under reduced pressure to obtain a product E-2 which was a white solid powder (1.35 g, 24.4%).

A solution of potassium permanganate (32.0 g, 202.8 mmol) dissolved in distilled water (48 ml) was divided into 6 parts and added to a three-neck round bottom flask containing Compound E-2 (4.6 g, 16.9 mmol) and pyridine (240 ml) every 30 minutes while increasing the temperature each time, and finally the temperature reached 90° C. The reaction mixture was stirred at 90° C. for 12 hours. The reaction mixture was filtered through a filter paper and washed with hot water/pyridine (v/v=3:1), and then the filtrate was concentrated under reduced pressure. The obtained product was added to the same round bottom flask, sodium hydroxide (4.0 g, 100 mmol), potassium permanganate (32.0 g, 202.8 mmol), and distilled water (150 ml) were added, and the reaction mixture was refluxed for 3 hours. The reaction was terminated by adding methanol to the reaction mixture, filtration was performed with a filter paper, and the filtrate was acidified by sulfuric acid to obtain a white solid product, which was filtered through a filter. The final product was dried under reduced pressure to obtain a solid powder product E-3 (3.30 g, 53.6%). Compound E-3 was sublimated under vacuum at 200° C. to obtain an acid anhydride compound 2.

### [Example 1] Preparation of Polyimide Resin (PI-1)

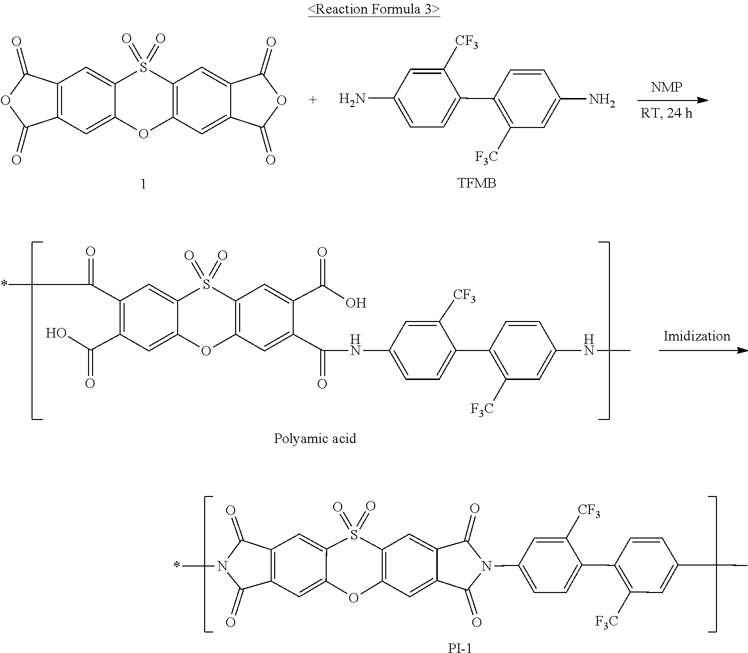

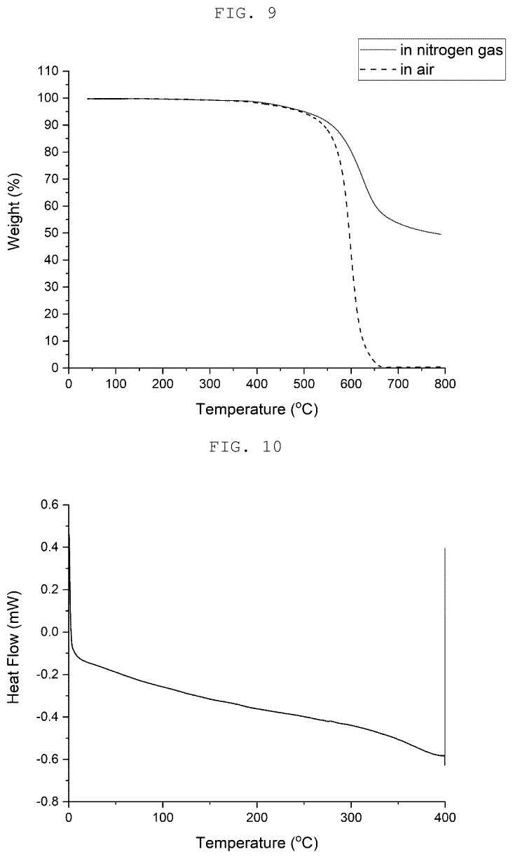

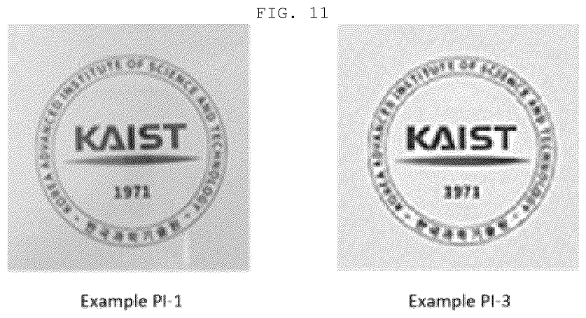

The dianhydride compound 1 (2.40 g, 6.45 mmol), a diamine monomer, 2 2′-bis(trifluoromethyl)benzidine (TFMB) (2.06 g, 6.44 mmol), and 45 mL of N-methyl-2-pyrrolidone (NMP) were added to a three-neck round flask to prepare a 10 wt % solution. A nitrogen inlet, an outlet, and a mechanical stirrer were connected to the round flask, and the prepared solution was stirred at room temperature for 24 hours to perform reaction, thereby preparing a polyamic acid. 1 mL of the prepared polyamic acid solution was applied on a glass substrate, and an imidization reaction was performed at 60° C. for 4 hours, at 110° C. for 24 hours, and at 350° C. for 2 hours, respectively, in a vacuum oven to prepare a polyimide film (PI-1). FIG. 7 is an NMR spectrum of the polyamic acid prepared according to Example 1. FIG. 8 is an FT-IR spectrum of the polyimide film prepared according to Example 1. FIG. 9 is a thermogravimetric analysis graph of the polyamide film (PI-1) prepared in Example 1 under a nitrogen (N₂) environment and an air (air) environment. FIG. 10 is a differential scanning calorimetric analysis graph of the polyimide film prepared in Example 1. The physical properties of the prepared polyimide film were measured by the measurement method described above, and are shown in the following Table 1.

### [Example 2] Preparation of Polyimide Resin (PI-2)

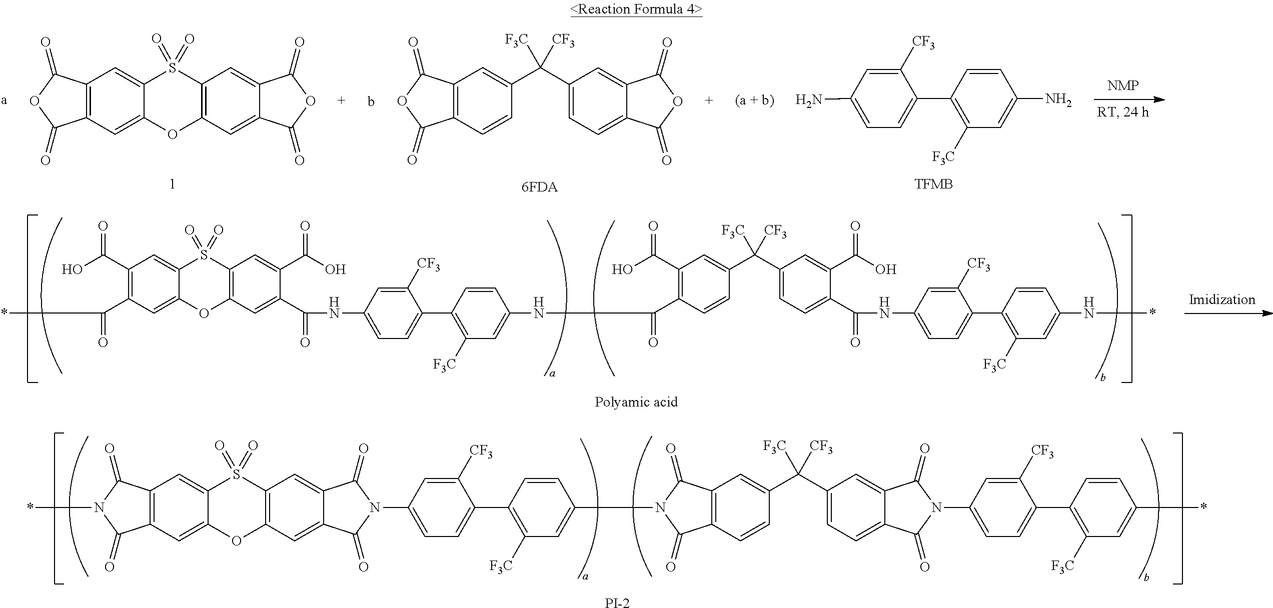

The dianhydride compound 1 (2.223 g, 5.80 mmol), 2,2-bis(4-aminophenyl) hexafluoropropane; 6FDA) (0.286 g, 0.644 mmol), a diamine monomer, 2 2′-bis(trifluoromethyl)benzidine (TFMB) (2.06 g, 6.44 mmol), and 45 mL of N-methyl-2-pyrrolidone (NMP) were added to a three-neck round flask to prepare a 10 wt % solution. A nitrogen inlet, an outlet, and a mechanical stirrer were connected to the round flask, and the prepared solution was stirred at room temperature for 24 hours to perform reaction, thereby preparing a polyamic acid (a:b=9:1). 1 mL of the prepared polyamic acid solution was applied on a glass substrate, and an imidization reaction was performed at 60° C. for 4 hours, at 110° C. for 24 hours, and at 350° C. for 2 hours, respectively, in a vacuum oven to prepare a polyimide film (PI-2). The physical properties of the prepared polyimide film were measured by the measurement method described above, and are shown in the following Table 1.

### [Example 3] Preparation of Polyimide Resin (PI-3)

The dianhydride compound 1 (1.917 g, 5.152 mmol), 2,2-bis(4-aminophenyl) hexafluoropropane; 6FDA) (0.572 g, 1.288 mmol), a diamine monomer, 2, 2′-bis(trifluoromethyl)benzidine (TFMB) (2.06 g, 6.44 mmol), and 45 mL of N-methyl-2-pyrrolidone (NMP) were added to a three-neck round flask to prepare a 10 wt % solution (a:b=8:2). A polyimide film was prepared in the same manner as in Example 2, except for the above. FIG. 11 is an image of the polyimide film prepared in Example 3. The physical properties of the prepared polyimide film were measured by the measurement method described above, and are shown in the following Table 1.

TABLE 1

| | λ₀ | T_{550 nm} | T_{d5} (° C.) | | | CTE (ppm/° C.) | | Yellow |
|---|---|---|---|---|---|---|---|---|
| | (nm) | (%) | In N₂ | In Air | T_{g} (° C.) | 2^{nd} run | 3^{rd} run | index |
| Example 1 | 349 | 88.9 | 500 | 492 | >400 | 1.98 | 2.10 | 6.0 |
| (PI-1) | | | | | | | | |
| Example 2 | 340 | 89.5 | 489 | 482 | >400 | 10.4 | 10.2 | 2.6 |
| (PI-2) | | | | | | | | |
| Example 3 | 338 | 90.0 | 488 | 480 | >400 | 14.6 | 14.3 | 2.2 |
| (PI-3) | | | | | | | | |

λ₀: cutoff wavelength: wavelength at which the film starts to show transmittance

T_{550 nm}: transmittance of film at 550 nm

T_{d5}: temperature at which the weight of the film is decreased by 5%, as a result of thermogravimetric analysis
T_{g}: glass transition temperature, not observed in a differential scanning calorimeter
CTE: coefficient of thermal expansion

As shown in Table 1, it was confirmed that the polyimide-based film of the present invention had a high transmittance and a low yellow index for a wavelength in a visible light region and transmitted light in a wide wavelength region. In addition, it was found that the film had a high thermal decomposition temperature under both nitrogen conditions and air conditions, had excellent heat resistance regardless of high-temperature environments, and a low coefficient of thermal expansion of 20 ppm/° C. or less.

Referring to FIGS. 9 and 10, the glass transition temperature (T_{g}) of Example 1 was 400° C. or higher, and decomposition did not occur even at a high temperature exceeding 400° C. In addition, referring to FIG. 11, it was confirmed that the polyimide-based film of the present invention was transparent, and the logo under the film was clearly visible.

That is, since the polyimide-based film of the present invention is colorless and transparent, and also, is not deformed even at a high-temperature process, the film may minimize a difference in expansion and shrinkage between dissimilar materials. Therefore, the polyimide-based film of the present invention is expected to be usefully applied as a flexible display device.

Novel polyimide-based resin and film including the acid anhydride compound and the dianhydride compound according to an exemplary embodiment of the present invention have excellent heat resistance, flexibility, and transparency. Specifically, the polyimide-based film according to an exemplary embodiment has a glass transition temperature of 400° C. or higher and a coefficient of thermal expansion of 20 ppm/° C. or less, and is transparent with a transmittance for a wavelength of 550 nm of 85% or more. The polyimide-based film may minimize differences in expansion and shrinkage between dissimilar materials by heat produced during the process and the operation. The polyimide-based film according to an exemplary embodiment may be used as a flexible display device.

Hereinabove, although the present invention has been described by specified matters and specific exemplary embodiments, they have been provided only for assisting in the entire understanding of the present invention, and the present invention is not limited to the exemplary embodiments. Various modifications and changes may be made by those skilled in the art to which the present invention pertains from the description.

Therefore, the spirit of the present invention should not be limited to the above-described exemplary embodiments, and the following claims as well as all modifications equal or equivalent to the claims are intended to fall within the scope and spirit of the invention.

What is claimed is:

1. A polyimide-based resin comprising a repeating unit represented by the following Chemical Formula 3 or 4: 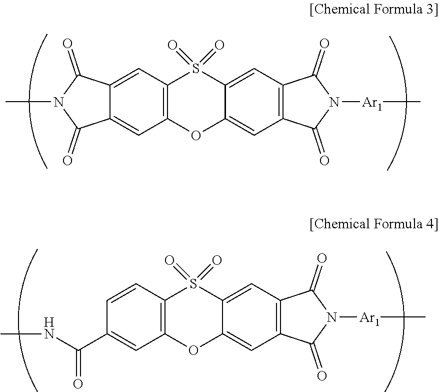
wherein Ar₁ is independently of each other 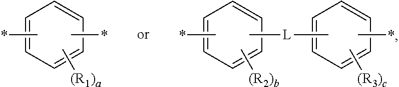
and
L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; a, b, and c are independently of one another an integer of 0 to 2.

2. The polyimide-based resin of claim 1, wherein the polyimide-based resin including the repeating unit represented by Chemical Formula 3 includes a repeating unit selected from the group consisting of the following Chemical Formulae 5 to 8: 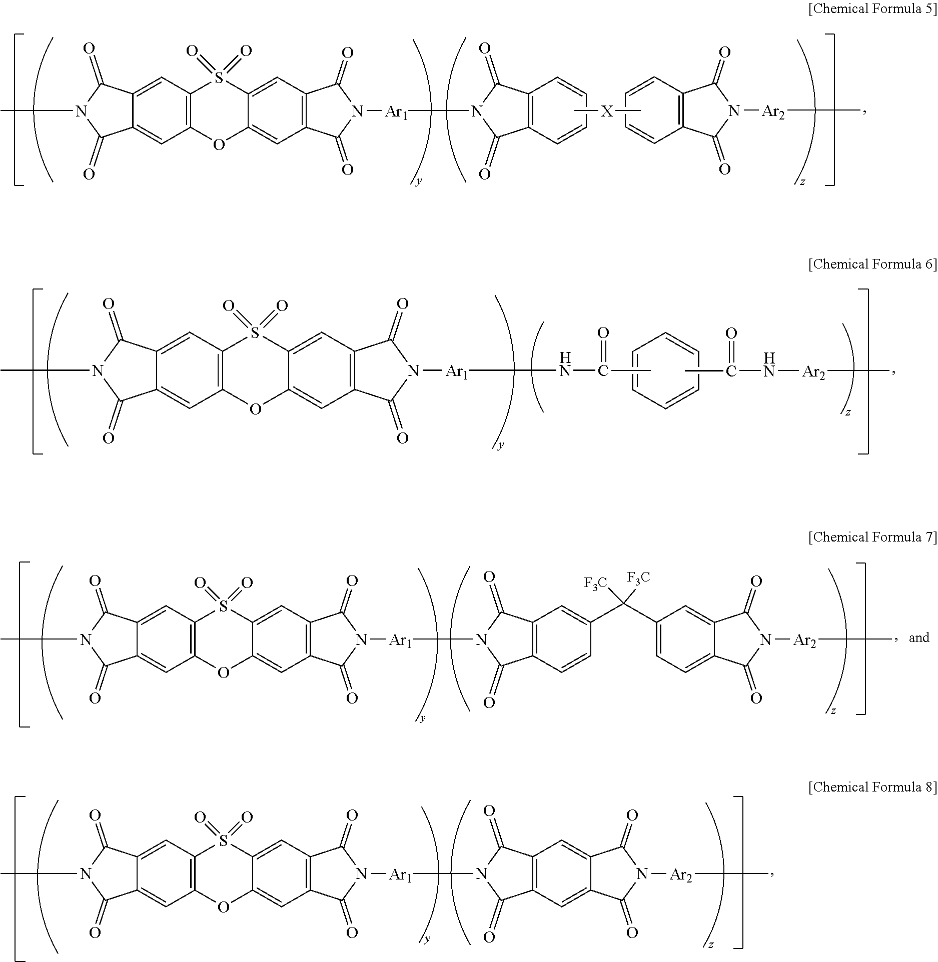
wherein X is a single bond, —CH₂—, —O—, —S—, —SO₂—, —CO—, isopropylidene, hexafluoroisopropylidene, or a combination thereof,
Ar₁ and Ar₂ are independently of each other 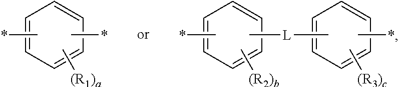
L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

3. The polyimide-based resin of claim 2, wherein Ar₁ and Ar₂ are independently of each other and selected from the group consisting of the following structures:

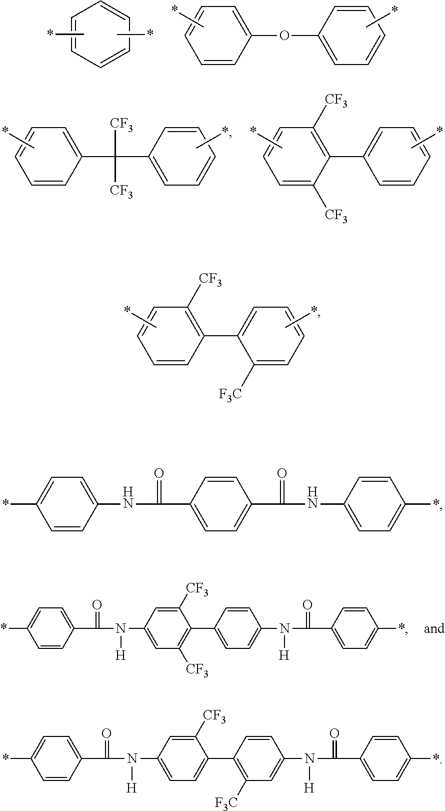

4. The polyimide-based resin of claim 3, wherein the polyimide-based resin includes a repeating unit represented by the following Chemical Formulae 9 to 11:

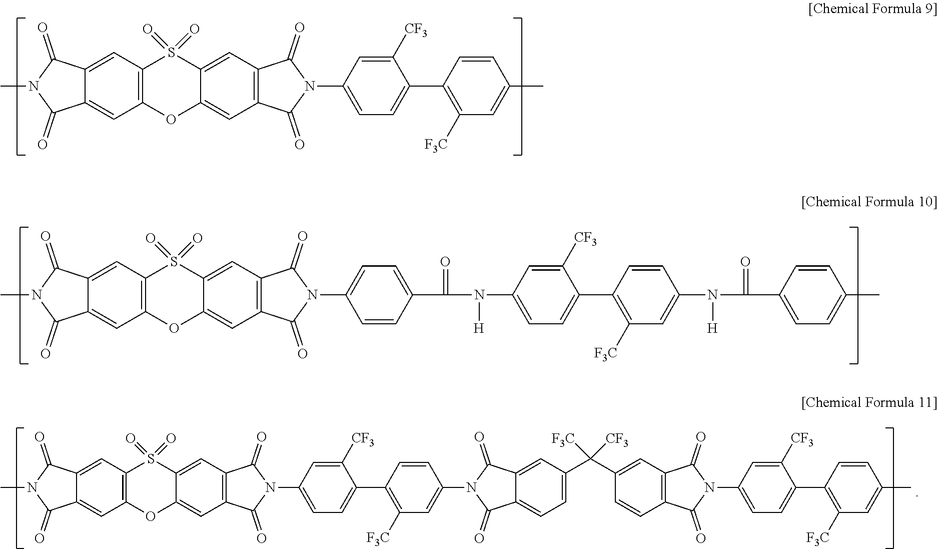

5. The polyimide-based resin of claim 1, wherein the polyimide-based resin including the repeating unit represented by Chemical Formula 4 includes a repeating unit represented by the following Chemical Formula 12: 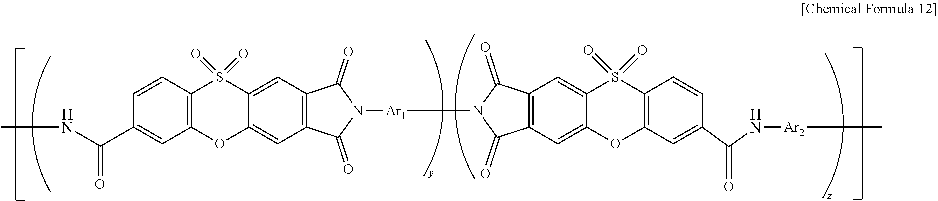
wherein Ar₁ and Ar₂ are independently of each other 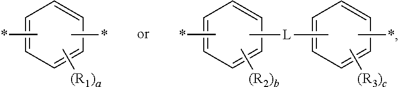
L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

6. The polyimide-based resin of claim 1, wherein the polyimide-based resin has a number average molecular weight of 10,000 to 200,000 g/mol.

7. A method for preparing a polyimide-based resin, the method comprising: reacting a dianhydride compound represented by the following Chemical Formula 1 and an aromatic diamine represented by the following Chemical Formula 21; sequentially reacting an aromatic acyl chloride represented by the following Chemical Formula 24 and an aromatic diamine represented by the following Chemical Formula 22; and performing imidization to prepare a polyimide-based resin including a repeating unit represented by the following Chemical Formula 6: 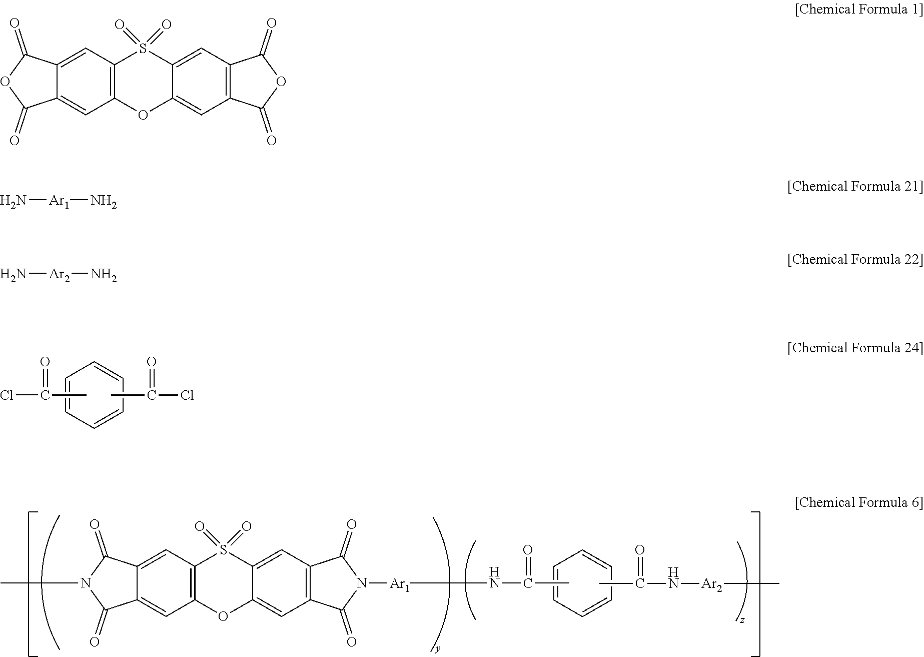
wherein Ar₁ and Ar₂ are independently of each other 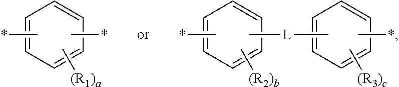
L is a single bond, —O—, —S—, —SO₂—, (C1-C7)alkylene, (C6-C12) arylene, or a combination thereof, and the arylene and the alkylene of L may be further substituted by one or more selected from (C1-C7)alkyl and halo(C1-C7)alkyl; R₁, R₂, and R₃ are independently of one another (C1-C7)alkyl, (C1-C7)alkoxy, or halo(C1-C7)alkyl; and a, b, and c are independently of one another an integer of 0 to 2, and
y:z is 100:0 to 10:90.

8. A composition for forming a polyimide-based film comprising the polyimide-based resin of claim 1.

9. The composition for forming a polyimide-based film of claim 8, further comprising an organic solvent.

10. The composition for forming a polyimide-based film of claim 8, wherein the composition for forming a polyimide-based film includes 5 to 50 wt % of the polyimide-based resin.

11. A polyimide-based film formed from the composition for forming a polyimide-based film of claim 8.

12. The polyimide-based film of claim 11, wherein the polyimide-based film has a glass transition temperature (T_{g}) of 400° C. or higher.

13. The polyimide-based film of claim 11, wherein the polyimide-based film has a coefficient of thermal expansion of 20 ppm/° C. or less.

14. The polyimide-based film of claim 11, wherein the polyimide-based film has a transmittance for a wavelength of 550 nm of 85% or more.

15. A flexible display device comprising the polyimide-based film of claim 11.

16. A polycyclic acid anhydride compound having a sulfonyl group represented by the following Chemical Formula 2: [chemical structure]
